SEQUENCE LISTING (32 sequences)

SEQ ID NO: 1 - DNA, 24 bp, Artificial Sequence
  synthetic construct; PP2A/C_5′ primer
gttcagcaacgagctggaccagtg

SEQ ID NO: 2 - DNA, 26 bp, Artificial Sequence
  synthetic construct; PP2A/C_3′ primer
ccaccacggtcatctggatctgacca

SEQ ID NO: 3 - DNA, 39 bp, Artificial Sequence
  synthetic construct; PP2A/Aalpha_5′ primer
acctctcagctgactgtcgggagaatgtgatcatgtccc

SEQ ID NO: 4 - DNA, 36 bp, Artificial Sequence
  synthetic construct; PP2A/Aalpha_3′ primer
ctccggactggccaagaccttggggatgattgtgga

SEQ ID NO: 5 - DNA, 42 bp, Artificial Sequence
  synthetic construct; PP2A/Abeta_5′ primer
cgatcgcggttttaatcgacgagctccgcaatgaagacgtgc

SEQ ID NO: 6 - DNA, 43 bp, Artificial Sequence
  synthtic construct; PP2A/Abeta_3′ primer
atactgacacaagcttccacagcaaggagggcgcactgaatcc

SEQ ID NO: 7 - DNA, 41 bp, Artificial Sequence
  synthetic construct; PPP2R2A_5′ primer
gctggaggagggaatgatattcagtggtgtttttctcaggt

SEQ ID NO: 8 - DNA, 40 bp, Artificial Sequence
  synthetic construct; PPP2R2B_3′ primer
gtaggatctctataccttccatcctcctctttcaagttat

SEQ ID NO: 9 - DNA, 33 bp, Artificial Sequence
  synthetic construct; PPP2R2B_5′ primer
gatcctgccaccatcacaaccctgcgggtgcct

SEQ ID NO: 10 - DNA, 32 bp, Artificial Sequence
  synthetic construct; PPP2R2B_3′ primer
gtcacacagccggattgtccctttgctgcggc

SEQ ID NO: 11 - DNA, 31 bp, Artificial Sequence
  synthetic construct; PPP2R2C_5′ primer
cdccdcccactcactcctgtccaccaacgat

SEQ ID NO: 12 - DNA, 38 bp, Artificial Sequence
  synthetic construct; PPP2R2C_3′ primer
ggacggcttgatgtccacgatgttgaagctcctgtcgg

SEQ ID NO: 13 - DNA, 35 bp, Artificial Sequence
  synthetic construct; PPP2R2D_5′ primer
tggtgcttctcgcaggtcaggggggccatcgacga

SEQ ID NO: 14 - DNA, 38 bp, Artificial Sequence
  synthetic construct; PPP2R2D_3′ primer
gacccgtagcgccgtgatcctaaatgggtctcgaagtc

SEQ ID NO: 15 - DNA, 42 bp, Artificial Sequence
  synthetic construct; PPP2R5A_5′ primer
gagtatgtttcaactaatcgtggtgtaattgttgaatcagcg

SEQ ID NO: 16 - DNA, 32 bp, Artificial Sequence
  synthetic construct; PPP2R5A_3′ primer
tcccataaattcggtgcagaacagtcttcagg

SEQ ID NO: 17 - DNA, 48 bp, Artificial Sequence
  synthetic construct; PPP2R5B-5′ primer
atggagacgaagctgccccctgcaagcacccccactagcccctcctcc

SEQ ID NO: 18 - DNA, 39 bp, Artificial Sequence
  synthetic construct; PPP2R5B_3′ primer
gacgggctcgatgaggacaccccgggtgctccccacact

SEQ ID NO: 19 - DNA, 33 bp, Artificial Sequence
  synthetic construct; PPP2R5C_5′ primer
cagtgacaacgcagcgaagattctgcccatcat

SEQ ID NO: 20 - DNA, 35 bp, Artificial Sequence
  synthetic construct; PPP2R5C_3′ primer
ctagcggccgtcctgggaggccagctcatcggccc

SEQ ID NO: 21 - DNA, 42 bp, Artificial Sequence
  synthetic construct; PPP2R5D_5′ primer
ggcccggcttaatccccagtatcccatgttccgagcccctcc

SEQ ID NO: 22 - DNA, 42 bp, Artificial Sequence
  synthetic construct; PPP2R5D_3′ primer
tcagagagcctcctggctggcagttaggaactcttccgcccg

SEQ ID NO: 23 - DNA, 38 bp, Artificial Sequence
  synthetic construct; PPP2R5E_5′ primer
ccggctattgtggcgttggtgtacaatgtgttgaaggc

SEQ ID NO: 24 - DNA, 48 bp, Artificial Sequence
  synthetic construct; PPP2R5E_3′ primer
taaagttggaattattccatcacgtctacgtctaagacctctctttaa

SEQ ID NO: 25 - DNA, 36 bp, Artificial Sequence
  synthetic construct; PPP2R3A_5′ primer
ggatgtggtggatacccaccctggtctcacgttcct

SEQ ID NO: 26 - DNA, 39 bp, Artificial Sequence
  synthetic construct; PPP2R3A_3′ primer
ctcatacatggagagtacaccgtctccatccacatccat

SEQ ID NO: 27 - DNA, 33 bp, Artificial Sequence
  synthetic construct; PPP2R3B_5′ primer
atgccgcccggcaaagtgctgcagccggtcctg

SEQ ID NO: 28 - DNA, 30 bp, Artificial Sequence
  synthetic construct; PPP2R3B_3′ primer
cgttcgtggggctggaggcggcgccaaggg

SEQ ID NO: 29 - DNA, 30 bp, Artificial Sequence
  synthetic construct; PPP2R3C_5′ primer
tcgtcggcgcctagcgacgcccaacacctg

SEQ ID NO: 30 - DNA, 45 bp, Artificial Sequence
  synthetic construct; PPP2R3C_3′ primer
atcgcttcctctccaatcataggtggtgtctggtgtttgtccagc

SEQ ID NO: 31 - DNA, 27 bp, Artificial Sequence
  synthetic construct; PPP2R4_5′ primer
gctgagggcgagcggcagccgccgcca

SEQ ID NO: 32 - DNA, 30 bp, Artificial Sequence
  synthetic construct; PPP2R4_3′ primer
gccagatgggtagggaccactgtggccacc

(19) United States
(12) Patent Application Publication
(10) Pub. No.: US 2014/0031291 A1
(43) Pub. Date: Jan. 30, 2014

(54) PP2A REGULATORY SUBUNIT MODIFICATION IN DISEASE

(71) Applicants: Peter Mohler, Powell, OH (US); Cynthia Carnes, Columbus, OH (US); Chenglong Li, Dublin, OH (US); Thomas Hund, Columbus, OH (US); Pui-Kai Li, Galloway, OH (US)

(72) Inventors: Peter Mohler, Powell, OH (US); Cynthia Carnes, Columbus, OH (US); Chenglong Li, Dublin, OH (US); Thomas Hund, Columbus, OH (US); Pui-Kai Li, Galloway, OH (US)

(73) Assignee: THE OHIO STATE UNIVERSITY, Columbus, OH (US)

(21) Appl. No.: 13/945,753

(22) Filed: Jul. 18, 2013

Related U.S. Application Data
(60) Provisional application No. 61/672,990, filed on Jul. 18, 2012.

(51) Int. Cl. C12Q 1/68

(52) U.S. Cl. CPC C12Q 1/6883
USPC 514/12.4; 435/6.12; 514/165

(57) ABSTRACT

Disclosed herein are methods for the diagnosis, detection, and treatment of cardiovascular disease and symptoms thereof in a subject. Disclosed herein are methods of diagnosing ischemic heart disease, non-ischemic heart disease, myocardial infarction, tachy-pacing induced non-ischemic heart disease, heart failure, and catecholaminergic-induced arrhythmia and symptoms thereof in a subject. Disclosed herein are methods of screening the efficacy of a pharmaceutical agent for treating cardiovascular disease and symptoms thereof in a subject. Disclosed herein are methods of identifying a subject eligible for a cardiovascular disease treatment clinical trial

### CROSS-REFERENCE To RELATED APPLICATIONS

This application claims benefit of U.S. Provisional Application No. 61/672,990, filed Jul. 18, 2012 and is hereby incorporated herein by reference in its entirety.

### STATEMENT REGARDING FEDERALLY SPONSORED RESEARCH

This invention was made with government support under Grant Nos. HL084583, HL083422, HL079031, HL62494, HL70250, HL089836, HL096805 awarded by the National Institutes of Health, Grant No. 0750891 awarded by the National Science Foundation and Grant No. W81XWH-10-1-0996 awarded by the U.S. Department of Defense. The government has certain rights in the invention.

### REFERENCE To SEQUENCE LISTING

The Sequence Listing submitted Jul. 18, 2013 as a text file named “26227_{—}0027U2_Sequence_Listing.txt,” created on Jul. 17, 2013, and having a size of 7,069 bytes is hereby incorporated by reference pursuant to 37 C.F.R. §1.52(e)(5).

### FIELD OF THE INVENTION

The invention relates generally to the diagnosis, detection, and treatment of cardiovascular disease and symptoms thereof in a subject. Specifically, the invention relates to a) methods of diagnosing ischemic heart disease, non-ischemic heart disease, myocardial infarction, tachy-pacing induced non-ischemic heart disease, heart failure, cardiac arrhythmias and catecholaminergic-induced arrhythmia and symptoms thereof in a subject, b) methods of screening the efficacy of a pharmaceutical agent for treating cardiovascular disease and symptoms thereof in a subject, and c) methods of identifying a subject eligible for a cardiovascular disease treatment clinical trial.

### BACKGROUND OF THE INVENTION

Dysregulation of protein phosphorylation has been linked to mechanical dysfunction and arrhythmias in cardiovascular disease states including, but not limited to, atrial fibrillation, sinus node disease, heart failure, and myocardial infarction [1-5]. Emphasis has been placed on defining the levels and activities of protein kinases in cardiovascular disease. Pharmacological inhibitors of kinase activity have enhanced the treatment of cardiovascular disease phenotypes [6-8]. Beta-adrenergic receptor blockers, for example, in the treatment of arrhythmia, hypertension, and heart failure function by dampening several kinase pathways. Protein phosphatases are also responsible for regulating protein phosphorylation levels, and new therapeutic targets related to this pathway offer additional therapeutic avenues. The expression, activity, and regulation of protein phosphatases with respect to cardiovascular disease offer an opportunity to realize these new therapeutics.

Protein phosphatase 2A (PP2A) is a serine/threonine phosphatase. The PP2A holoenzyme is comprised of three subunits: scaffolding (A subunit), regulation (B subunit), and catalytic activity (C subunit). Thus, the diversity of PP2A cellular function arises from specific combinatorial holoenzyme products (2 A subunit genes, 13 B subunit genes, and 2 C subunit genes) [9]. PP2A function is critical for the regulation of targets in both excitable and non-excitable cells including, but not limited to, ion channels and transporters, regulatory enzymes, transcription factors, and cytoskeletal proteins. Moreover, PP2A is linked to human disease. For example, PP2A dysfunction is linked with oncogenic regulation and Alzheimer's Disease [10, 11], and the design of PP2A-based inhibitors is a major area of research for cancer therapies. In myocytes, PP2A activity is linked with multiple targets important in membrane excitability and excitation-contraction coupling including ryanodine receptor (RyR2), connexin 43, Caᵥ1.2, troponin, Na/Ca exchanger (NCX), and phospholamban [12-18]. The manipulation of PP2A activity or expression in animal or cell models produces defects in myocyte physiology and cardiac phenotypes [15, 19]. What is needed in the art are compositions and methods that determine expression of the PP2A genes, thereby detecting heart disease, and the identification targets for treatment for heart failure and arrhythmia.

### SUMMARY OF THE INVENTION

Disclosed herein is a method of detecting heart disease in a subject, comprising determining in a sample from a subject expression levels of one or more Protein Phosphatase 2A (PP2A) genes and comparing the expression level of the one or more PP2A genes to expression levels of corresponding PP2A genes in a sample from a normal control, wherein an increase or decrease in the expression levels of one or more PP2A genes in the sample from the subject compared to the expression levels of corresponding PP2A genes in the sample from the normal control detects heart disease in the subject.

Also disclosed is a method of detecting heart failure in a subject, comprising determining in a sample from a subject an increased ratio of phosphorylated catalytic subunit genes to total catalytic subunit genes compared to a ratio of phosphorylated catalytic subunit genes to total catalytic subunit genes in a normal control sample, wherein the increased ratio of phosphorylated catalytic subunit genes to total catalytic subunit genes in the subject detects heart failure.

Further disclosed is a method of detecting heart failure in a subject, comprising determining in a sample from a subject a decreased ratio of methylated catalytic subunit genes to total catalytic subunit genes compared to a ratio of methylated catalytic subunit genes to total catalytic subunit genes in a normal control sample, wherein the decreased ratio of methylated catalytic subunit genes to total catalytic subunit genes in the subject detects heart failure.

Disclosed herein is a method of determining responsiveness of a heart disease in a subject to treatment with a pharmaceutical agent, comprising a) determining the expression levels of one or more PP2A genes in a first sample from the subject before treatment; b) administering to the subject the pharmaceutical agent; c) determining the expression levels of the corresponding PP2A genes in a second sample from the subject after treatment; and d) comparing the expression levels of the one or more PP2A genes in the second sample from the subject to the expression levels of the corresponding PP2A genes in the first sample from the subject, wherein the normalization of the expression levels of PP2A genes in the second sample from the subject indicates responsiveness of the heart disease to the pharmaceutical agent.

Also disclosed is a method of screening the efficacy of a pharmaceutical agent for treating heart disease in a subject, comprising a) determining the expression levels of one or more PP2A genes in a first sample from the subject before treatment; b) administering to the subject the pharmaceutical agent; c) determining the expression levels of the corresponding PP2A genes in a second sample from the subject after treatment; and d) comparing the expression levels of the one or more PP2A genes in the second sample from the subject to the expression levels of the corresponding PP2A genes in the first sample from the subject, wherein the normalization of the expression levels of PP2A genes in the second sample from the subject indicates efficacy of the pharmaceutical agent.

Further disclosed is a method of identifying a subject appropriate for a heart disease treatment clinical trial comprising determining in a sample from the subject the expression levels of one or more PP2A genes, wherein the expression levels of the one or more PP2A genes is indicative of heart disease, indicates whether the subject is appropriate for the treatment clinical trial.

Further disclosed is a method of identifying a subject appropriate for a heart disease treatment clinical trial comprising determining in a sample from a subject an increased ratio of phosphorylated catalytic subunit genes to total catalytic subunit genes compared to a ratio of phosphorylated catalytic subunit genes to total catalytic subunit genes in a normal control sample, wherein the increased ratio of phosphorylated catalytic subunit genes to total catalytic subunit genes in the subject indicates that the subject is appropriate for the treatment clinical trial.

Also disclosed is a method of identifying a subject appropriate for a heart disease treatment clinical trial comprising determining in a sample from a subject a decreased ratio of methylated catalytic subunit genes to total catalytic subunit genes compared to a ratio of methylated catalytic subunit genes to total catalytic subunit genes in a normal control sample, wherein the decreased ratio of methylated catalytic subunit genes to total catalytic subunit genes in the subject indicates that the subject is appropriate for the treatment clinical trial.

### BRIEF DESCRIPTION OF THE FIGURES

The accompanying figures, which are incorporated in and constitute a part of this specification, illustrate several aspects and together with the description serve to explain the principles of the invention.

FIG. 1 shows the distribution of the PP2A subunits in human heart.

FIG. 2 shows the differential subcellular expression of PP2A regulatory subunits in primary adult cardiomyocytes.

FIG. 3 shows the differential regulation of PP2A regulatory subunits in ischemic heart failure.

FIG. 4 shows PP2A subunit regulation in canine cardiovascular disease models.

FIG. 5 shows the post-translational regulation of cardiac PP2A function in human cardiovascular disease.

FIG. 6 shows detailed gene and protein nomenclature of the PP2A subunit families.

FIG. 7 shows PP2A subunit regulation in a mouse model of human catecholaminergic-induced arrhythmia.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention can be understood more readily by reference to the following detailed description of the invention and the Examples included therein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### A. Definitions and Nomenclature

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein disclosed as “about” that particular value in addition to the value itself For example, if the value “10” is disclosed, then “about 10” is also disclosed. It is also understood that when a value is disclosed that is “less than or equal to” the value, “greater than or equal to the value” and possible ranges between values are also disclosed, as appropriately understood by the skilled artisan. For example, if the value “10” is disclosed, the “less than or equal to 10” as well as “greater than or equal to 10” is also disclosed. It is also understood that throughout the application, data are provided in a number of different formats and that these data represent endpoints, starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Optional” or “optionally” means that the subsequently described event or circumstance may or may not occur and that the description includes instances where said event or circumstance occurs and instances where it does not.

The word “or” as used herein means any one member of a particular list and also includes any combination of members of that list.

By “modulate” is meant to alter, by increasing or decreasing.

By “normal subject” is meant an individual who does not have heart disease.

By “normal control sample” is meant a reference sample obtained from one or more normal subjects.

As used herein, “appropriate for a treatment clinical trial” means that a subject is suitable for inclusion in the treatment clinical trial.

As used herein, the term “subject” and “patient” can be used interchangeably.

The amino acid abbreviations used herein are conventional one letter codes for the amino acids and are expressed as follows: A, alanine; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; E, glutamic acid.

“Peptide” as used herein refers to any peptide, oligopeptide, polypeptide, gene product, expression product, or protein. For example, a peptide can be a receptor. A polypeptide is comprised of consecutive amino acids. The term “polypeptide” encompasses naturally occurring or synthetic molecules.

In addition, as used herein, the term “peptide” or “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “treat” is meant to administer a compound or molecule to a subject, such as a human or other mammal (for example, an animal model), that has a condition or disease, such as heart disease (for example), an increased susceptibility for developing such a disease, in order to prevent or delay a worsening of the effects of the disease or condition, or to partially or fully reverse the effects of the disease. To “treat” can also refer to non-pharmacological methods of preventing or delaying a worsening of the effects of the disease or condition, or to partially or fully reversing the effects of the disease. For example, “treat” can mean a course of action to prevent or delay a worsening of the effects of the disease or condition, or to partially or fully reverse the effects of the disease other than by administering a compound.

By “prevent” is meant to minimize the chance that a subject who has susceptibility for developing disease such as heart disease will develop such a disease, or one or more symptoms associated with the disease.

By “probe,” “primer,” or “oligonucleotide” is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for PP2A nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled either radioactively or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction (PCR), single stranded conformational polymorphism (SSCP) analysis, restriction fragment polymorphism (RFLP) analysis, Southern hybridization, Northern hybridization, in situ hybridization, and electrophoretic mobility shift assay (EMSA). One or more of the primers or probes described herein can be used to amplify or detect one or more of the PP2A genes or gene products described herein. Additionally, the primers and probes described herein can be used in the methods described herein.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The nucleic acids, such as, the polynucleotides described herein, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 3rd Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1Plus DNA synthesizer. Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### B. Compositions

The full characterization of the PP2A family of polypeptides in the heart is disclosed herein. The expression of seventeen different PP2A genes in human heart and the differential expression and distribution of these genes across multiple species and different cardiac chambers were defined. The subcellular distributions of PP2A regulatory subunits in myocytes were also defined. Differential regulation of PP2A scaffolding, regulatory, and catalytic subunit expression in multiple models of cardiovascular disease as well as in human heart failure samples was found. PP2A regulation in disease extends beyond expression and subcellular location, linking differential post-translational modifications of the PP2A holoenzyme in human heart failure. Together, these findings provide new insight into the functional complexity of PP2A expression, activity, and regulation in heart and in human cardiovascular disease and identify targets for the treatment of heart failure and arrhythmia.

Disclosed herein is a gene expression panel or array for detecting heart disease, said panel or array consisting of primers or probes capable of measuring expression levels of a statistically significant number of genes of one or more of the following genes: PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, and PPP2R4. Any of the primers or probes described herein can be used in the gene expression panels or arrays for detecting heart disease as described herein. The gene expression panel or array can consist of primers or probes capable of detecting one or more genes disclosed herein. Examples of such primers and probes can be found in Table 1 of Example 1. This list is not intended to be limiting, as one of skill in the art can readily identify primers and probes for use with the compositions and methods disclosed herein, but are intended to be exemplary.

The profile of the expression levels of the genes can be used to compute a statistically significant value based on differential expression of the group of genes, wherein the computed value correlates to a diagnosis of heart disease. The variance in the obtained profile of expression levels of the said selected genes or gene expression products can be either upregulated or downregulated as compared to a control. This is described in more detail herein.

By “heart disease” is meant ischemic heart disease, non-ischemic heart disease, myocardial infarction, tachy-pacing induced and non-ischemic heart disease, cardiac arrhythmias or catecholaminergic-induced arrhythmia.

Also disclosed are diagnostic kits containing probes or primers for measuring the expression of one or more of the genes disclosed herein. For example, disclosed are diagnostic kits containing probes or primers for measuring the expression of one or more of the mRNA or protein products of: PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4. The kits can consist of primers or probes capable of detecting one or more genes disclosed herein. Examples of such primers and probes can be found in Table 1 of Example 1. This list is not intended to be limiting, as one of skill in the art can readily identify primers and probes for use with the compositions and methods disclosed herein, but are intended to be exemplary.

Disclosed herein are solid supports comprising one or more primers, probes, polypeptides, or antibodies capable of hybridizing or binding to one or more of the PP2A genes, such as PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4. Solid supports are solid-state substrates or supports with which molecules, such as analytes and analyte binding molecules, can be associated. Analytes, such as calcifying nano-particles and proteins, can be associated with solid supports directly or indirectly. For example, analytes can be directly immobilized on solid supports. Analyte capture agents, such a capture compounds, can also be immobilized on solid supports. The solid supports can consist of primers or probes capable of detecting one or more genes disclosed herein. Examples of such primers and probes can be found in Table 1 of Example 1. This list is not intended to be limiting, as one of skill in the art can readily identify primers and probes for use with the compositions and methods disclosed herein, but are intended to be exemplary.

The term “differentially expressed” or “differential expression,” as well as the term “variant,” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example, as measured by the amount of messenger RNA transcript and/or the amount of protein in a sample as compared with the measurable expression level of a given biomarker in a normal control. In one embodiment, the differential expression can be compared using the ratio of the level of expression of a given biomarker or biomarkers as compared with the expression level of the given biomarker or biomarkers of a normal control, wherein the ratio is not equal to 1.0. Thus, an RNA or protein is differentially expressed if the ratio of the level of expression in a first sample as compared with a second sample is greater than 1.0, for example, a ratio of greater than 1, 1.2, 1.5, 1.7, 2, 3, 3, 5, 10, 15, 20 or more. Further, an RNA or protein is differentially expressed if the ratio of the level of expression in a first sample as compared with a second sample is less than 1.0., for example, less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the normal control or reference profile. For example, similarity can refer to a fold difference compared to a normal control. In one example, there is no statistically significant difference in the level of expression of the biomarkers.

The phrase “determining the expression of biomarkers” as used herein refers to determining or quantifying RNA or proteins or protein activities or protein-related metabolites expressed by the genes disclosed herein. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein,” it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses.

Accordingly, in one example, the biomarker expression levels are determined using arrays, optionally microarrays, RT-PCR, optionally quantitative RT-PCR, nuclease protection assays or Northern blot analyses.

A form of solid support is an array. Another form of solid support is an array detector. An array detector is a solid support to which multiple different capture compounds or detection compounds have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In preferred embodiments, a multiwell glass slide can be employed that normally contains one array per well. This feature allows for greater control of assay reproducibility, increased throughput and sample handling, and ease of automation.

Different compounds can be used together as a set. The set can be used as a mixture of all or subsets of the compounds used separately in separate reactions, or immobilized in an array. Compounds used separately or as mixtures can be physically separable through, for example, association with or immobilization on a solid support. An array can include a plurality of compounds immobilized at identified or predefined locations on the array. Each predefined location on the array generally can have one type of component (that is, all the components at that location are the same). Each location will have multiple copies of the component. The spatial separation of different components in the array allows separate detection and identification of the polynucleotides or polypeptides disclosed herein.

It is not required that a given array be a single unit or structure. The set of compounds may be distributed over any number of solid supports. For example, at one extreme, each compound may be immobilized in a separate reaction tube or container, or on separate beads or microparticles. Different modes of the disclosed method can be performed with different components (for example, different compounds specific for different proteins) immobilized on a solid support.

Some solid supports can have capture compounds, such as antibodies, attached to a solid-state substrate. Such capture compounds can be specific for calcifying nano-particles or a protein on calcifying nano-particles. Captured calcifying nano-particles or proteins can then be detected by binding of a second detection compound, such as an antibody. The detection compound can be specific for the same or a different protein on the calcifying nano-particle.

Methods for immobilizing nucleic acids, peptides or antibodies (and other proteins) to solid-state substrates are well established Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy]succinimide ester (GMBS). These and other attachment agents, as well as methods for their use in attachment, are described in Protein immobilization: fundamentals and applications, Richard F. Taylor, ed. (M. Dekker, New York, 1991);, Johnstone and Thorpe, Immunochemistry In Practice (Blackwell Scientific Publications, Oxford, England, 1987) pages 209-216 and 241-242, and Immobilized Affinity Ligands; Craig T. Hermanson et al., eds. (Academic Press, New York, 1992) which are incorporated by reference in their entirety for methods of attaching antibodies to a solid-state substrate. Antibodies can be attached to a substrate by chemically cross-linking a free amino group on the antibody to reactive side groups present within the solid-state substrate. For example, antibodies may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS3) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each of the components (compounds, for example) immobilized on the solid support preferably is located in a different predefined region of the solid support. Each of the different predefined regions can be physically separated from each other of the different regions. The distance between the different predefined regions of the solid support can be either fixed or variable. For example, in an array each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Optionally, at least one address on the solid support can be a probe specific for one or more of the genes disclosed herein. Disclosed are solid supports where at least one address is the sequences or portion of sequences set forth in any of the peptide sequences disclosed herein. Solid supports can also contain at least one address that is a variant of the sequences or part of the sequences set forth in any of the nucleic acid sequences disclosed herein. Solid supports can also contain at least one address that is a variant of the sequences or portion of sequences set forth in any of the peptide sequences disclosed herein.

In addition, the genes described herein may be used as markers for presence or progression of heart disease. The methods and assays described elsewhere herein may be performed over time, and the change in the level of reactive polypeptide(s), polynucleotide(s), and or polypeptide “activity” evaluated. For example, the assays may be performed every 24-72 hours for a period of 6 months to 1 year, and thereafter performed as needed. Assays can be performed prior to, during, or after a treatment protocol. As noted herein, to improve sensitivity, multiple genes may be assayed within a given sample. Binding agents specific for different proteins, antibodies, nucleic acids thereto provided herein may be combined within a single assay. Further, multiple primers or probes may be used concurrently. The selection of receptors may be based on routine experiments to determine combinations that results in optimal sensitivity. To assist with such assays, specific biomarkers can assist in the specificity of such tests. As such, disclosed herein is a biomarker, wherein the biomarker is capable of binding to or hybridizing with one or more genes or peptides as disclosed herein.

According to a further aspect, there is provided a computer implemented product for detecting heart disease, comprising (a) a means for receiving values corresponding to a subject expression profile in a subject sample; and (b) a database comprising a reference expression profile associated with a diagnosis or prognosis, wherein the subject biomarker expression profile and the biomarker reference profile each have at least three values representing the expression level of at least one biomarker selected from PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4, wherein the computer implemented product selects the biomarker reference expression profile most similar to the subject biomarker expression profile, to thereby diagnose or prognose the subject.

Preferably, a computer implemented product described herein is for use with a method described herein.

Preferably, the data structure is capable of configuring a computer to respond to queries based on records belonging to the data structure, each of the records comprising: (a) a value that identifies a biomarker reference expression profile of at least one gene selected from PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 and (b) a value that identifies the probability of a diagnosis associated with the biomarker reference expression profile.

According to a further aspect, there is provided a computer system comprising (a) a database including records comprising a biomarker reference expression profile of at least one gene selected from PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4, associated with a diagnosis of heart disease; (b) a user interface capable of receiving a selection of gene expression levels of the at least one gene for use in comparing to the biomarker reference expression profile in the database; and (c) an output that displays a diagnosis or prognosis or therapy according to the biomarker reference expression profile most similar to the expression levels of the at least one gene.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

### C. Methods

The disclosed genes and peptides, including PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 can be used in a variety of different methods, for example in prognostic, predictive, diagnostic, and therapeutic methods and as a variety of different compositions.

Disclosed herein is a method of detecting heart disease in a subject, comprising determining in a sample from a subject expression levels of one or more PP2A genes and comparing the expression level of the one or more PP2A genes to expression levels of corresponding PP2A genes in a sample from a normal control, wherein an increase or decrease in the expression levels of one or more PP2A genes in the sample from the subject compared to the expression levels of corresponding PP2A genes in the sample from the normal control detects heart disease in the subject.

The expression level of the one or more PP2A genes is determined by amplifying a nucleic acid sample obtained from the subject. The method can also further comprise administering a therapeutic composition to the subject.

The PP2A genes can be catalytic subunit genes, such as PPP2CA or PPP2CB. One can measure an increase in the expression level of PPP2CA and PPP2CB in the subject compared to the expression levels of PPP2CA and PPP2CB in the normal control to detect heart disease in the subject. One can then further administer a therapeutic composition to the subject.

The PP2A genes can also be scaffolding subunit genes, such as PPP2R1A or PPP2R1B. Disclosed herein is a method of determining an increase in the expression level of PPP2R1A and PPP2R1B in the subject compared to the expression levels of PPP2R1A and PPP2R1B in the normal control thereby detecting heart disease in the subject. One can then further administer a therapeutic composition to the subject.

The PPP2A genes can be regulatory subunit genes, such as PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4. Disclosed herein is a method of determining increased expression levels of one or more of PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, and PPP2R4 in the subject compared to the expression levels of the corresponding genes in the normal control detect heart disease. One can then further administer a therapeutic composition to the subject. One can determine the expression levels of one or more of PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, and PPP2R4 in the subject compared to the expression levels of the corresponding genes in the normal control to detect heart disease. One can then administer a therapeutic composition to the subject.

Also disclosed herein is a method wherein increased expression levels of PPP2CA, PPP2CB, PPP2R5A, PPP2R5B, PPP2R5E, PPP2R3A, and PPP2R4 compared to the expression levels of the corresponding genes in the normal control detects ischemic heart disease in the subject. One can then administer a therapeutic composition to the subject.

Also disclosed is a method of determining an increased level of expression of PPP2CA, PPP2CB, PPP2R1A, PPP2R1B, PPP2R5A, PPP2R5B, PPP2R5E, PPP2R3A, and PPP2R4 compared to the expression levels of the corresponding genes in the normal control to detect non-ischemic heart disease in the subject. One can then administer a therapeutic composition to the subject.

Also disclosed is a method of determining increased levels of expression of PPP2CA, PPP2CB, PPP2R1A, PPP2R1B, PPP2R5D, and PPP2R4, and a decreased level of expression of PPP2R5A compared to the expression levels of the corresponding genes in the normal control to detect myocardial infarction in the subject. One can then administer a therapeutic composition to the subject.

The heart disease can be tachy-pacing induced non-ischemic heart disease, for example. Thus, disclosed is a method of determining increased levels of expression of PPP2CA, PPP2CB, PPP2R1A, PPP2R1B, PPP2R2B, PPP2R5B, PPP2R5D, PPP2R5E, and PPP2R4 compared to the expression levels of the corresponding genes in the normal control to detect tachy-pacing induced non-ischemic heart disease in the subject. One can then administer a therapeutic composition to the subject.

The heart disease can be catecholaminergic induced arrhythmia. Thus, disclosed is a method of determining increased levels of expression of PPP2CA, PPP2CB, PPP2R1A, PPP2R1B, PPP2R5A, and PPP2R5B, and a decreased level of expression of PPP2R5E compared to the expression levels of the corresponding genes in the normal control to detect cardiac arrhythmias or catecholaminergic induced arrhythmia in the subject. One can then administer a therapeutic composition to the subject.

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, frozen sections taken for histological purposes, blood, blood fraction, plasma, serum, sputum, stool, tears, transudates, exudates, breast milk, prostatic fluid, semen, mucus, hair, skin, urine, effusions, ascitic fluid, amniotic fluid, saliva, cerebrospinal fluid, cervical secretions, vaginal secretions, endometrial secretions, gastrointestinal secretions, bronchial secretions, cell line, tissue sample, or secretions. A biological sample may be provided by removing a sample of cells from a subject but can also be accomplished by using previously isolated cells (e.g., isolated by another person, at another time, and/or for another purpose). Archival tissues, such as those having treatment or outcome history, may also be used. Biological samples also include explants and primary and/or transformed cell cultures derived from animal or human tissues.

As used herein, the term “subject” refers to a mammal, including both human and other mammals. The methods disclosed herein can be applied to human subjects.

Also disclosed is a method of detecting heart failure in a subject, comprising determining in a sample from a subject an increased ratio of phosphorylated catalytic subunit genes to total catalytic subunit genes compared to a ratio of phosphorylated catalytic subunit genes to total catalytic subunit genes in a normal control sample, wherein the increased ratio of phosphorylated catalytic subunit genes to total catalytic subunit genes in the subject detects heart failure.

The ratio of phosphorylated catalytic subunit genes to total catalytic subunit genes can be determined by performing measurements of each form using selective antibodies versus ratios of total protein levels and housekeeping “control” polypeptides by immunoblot.

Also disclosed is a method of detecting heart failure in a subject, comprising determining in a sample from a subject a decreased ratio of methylated catalytic subunit genes to total catalytic subunit genes compared to a ratio of methylated catalytic subunit genes to total catalytic subunit genes in a normal control sample, wherein the decreased ratio of methylated catalytic subunit genes to total catalytic subunit genes in the subject detects heart failure. The ratio of methylated catalytic subunit genes to total catalytic subunit genes can be determined by performing measurements of each form using selective antibodies versus ratios of total protein levels and housekeeping “control” polypeptides by immunoblot.

As the term is used herein, an “increased ratio” refers to an identified, reproducible, quantitative or qualitative, increase in the relative amount of the physical presence of one or more of the following: PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4, as compared with another target or with housekeeping genes including, but not limited to cardiac gene products including actin and GAPDH that display minimal to no change in heart disease phenotypes. Housekeeping gene products may differ based on the specific cardiac pathology.

As the term is used herein, a “decreased ratio” refers to an identified, reproducible, quantitative or qualitative, decrease in the relative amount of the physical presence of one or more of the following: PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4, as compared with another target or with housekeeping genes including, but not limited to cardiac gene products including actin and GAPDH that display minimal to no change in heart disease phenotypes. Housekeeping gene products may differ based on the specific cardiac pathology.

The relative amount of the respective entities is determined by quantitative measurement of the two entities (e.g., a specifically phosphorylated OR methylated molecules in a target cell or tissue) by appropriately comparable methods of detection, with calculation of the relative ratio by standard mathematical means (e.g., determination of the ratio of the two determined measurements) to produce a quantitative measurement reflective of the relative amount of the two entities. The identical methods are used to measure the relative ratio of the same respective entities in an appropriate control cell or tissue, to produce a quantitative measurement reflective of the relative amount of the two entities under normal, non-disease conditions. An increase in the relative ratio is identified by comparison of the relative amount calculated from the target tissue to the relative amount calculated from the normal control tissue, wherein a reproducible, statistically significant, higher relative amount is quantitatively or qualitatively determined. A decrease in the relative ratio is identified by comparison of the relative amount calculated from the target tissue to the relative amount calculated from the normal control tissue, wherein a reproducible, statistically significant, lower relative amount is quantitatively or qualitatively determined.

It will be appreciated that in the methods described herein that involve comparison of a determined level or amount to a reference or normal control amount, standard methods available to the skilled practitioner can be used to obtain the appropriate reference amounts. In one embodiment, the reference amount is obtained from a reference or control sample obtained from the subject (e.g. a biological sample). Typically, normal, healthy tissue, (e.g., located adjacent to the sample tissue), is used as the reference profile or normal control tissue. Preferably the control tissue, or biological sample, is of the same type as the tested sample tissue. In certain embodiments, wherein the progression of heart disease in a subject is to be monitored over time, the reference or control sample can be the tested sample tissue taken from the subject at an earlier date.

In some embodiments, the reference can be a level of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 in a normal healthy subject with no symptoms or signs of heart disease. Preferably the reference is of the same tissue type as the tested sample. For example, a normal healthy subject has normal heart tissue morphology, and/or is not diagnosed with heart disease, and/or has been identified as having a genetic predisposition for developing heart disease (e.g. a family history of heart disease or a genetic test for genotypes associated with an increased risk of heart disease) and/or has been exposed to risk factors for heart disease (e.g. smoke, cigarette smoke, alcohol, tobacco, obesity, etc). In some embodiments, the reference can also be a level of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 in a control sample, a pooled sample of control individuals or a numeric value or range of values based on the same.

Methods to measure, detect or determine PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 are well known to a skilled artisan. The genes or the encoded proteins can be measured or detected. For example, an increase in PPP2CA can be determined by measuring the PPP2CA gene expression levels or the PPP2CA protein levels. Any of the disclosed methods of detecting heart disease in a subject, detecting heart failure in a subject, determining responsiveness of a subject to a treatment, screening for efficacy of pharmaceutical agents, and identifying subjects for clinical trial can use the methods or assays of measuring or detecting the PP2A genes or proteins described herein.

Methods of measuring or detecting gene expression are well known in the art. Assays such as but not limited to reverse transcription-PCR, real time PCR, and northern blots can be used. Reverse transcription-PCR allows for the amplification of cDNA produced from RNA from a particular sample. The RNA reflects the gene expression and is ultimately amplified after being converted to cDNA. For example, gene expression can be measured by obtaining a sample containing mRNA from a subject. Using reverse transcriptase, the mRNA can be converted to cDNA, thus making a complimentary DNA strand. The cDNA can then be amplified using common amplification techniques. Real-time PCR allows for the direct quantification of DNA in real time (i.e. during amplification). Northern blots provide the ability to determine the presence and quantification of RNA in a sample by hybridizing the RNA with a specific probe that binds to the nucleic acid sequence of interest. The primers and probes used to detect specific nucleic acids (RNA, DNA or cDNA) can be determined by one of skill in the art based on the sequence of the gene to be detected. For example, the primers used for reverse transcription-PCR can be short nucleic acid sequences that are complementary to sequences of a particular gene, such as PPP2CA. Any of the primers identified in Table 1 can be used to detect or measure gene expression of the PP2A genes.

In one embodiment, methods of measurement involve use of a binding protein (e.g. an antibody) for specific detection (e.g., immunodetection) of the specified protein. Such methods to measure protein level include ELISA (enzyme linked immunosorbent assay), Western blot, proteomic microarray, immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques. Such antibody-based methods can distinguish between the proteins disclosed herein. Antibodies specific for the various isoforms of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 are available in the art.

The techniques of immunohistochemistry (“IHC”) and immunocytochemistry (“ICC”) are particularly suitable for use in the methods described herein. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that will undergo a biochemical reaction and thereby experience a change (e.g., color or light emission), upon encountering the targeted molecules. In some instances, signal amplification can be integrated into the particular protocol, wherein a secondary antibody, which includes the marker stain or marker signal, follows the application of a primary specific antibody. In some embodiments, the level of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 can be determined using a staining intensity scale and immunohistochemistry as described in the Examples herein.

The technique of proteomic microarray analysis can be used in the methods described herein. Proteomic microarrays are based on miniature arrays of ligands. Proteins in a sample bind to the ligands on the array and are detected and the amount quantified, often by using fluorescent tags (Templin et al. Proteomics 2003 3:2155-2166; MacBeath, Nature Genetics 2002 32:526-532).

In addition, protein levels may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000). In certain embodiments, a gas phase ion spectrophotometer is used.

In other embodiments, laser-desorption/ionization mass spectrometry is used to analyze the level of a protein. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. Detection of the presence of AID mRNA or protein will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis, etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Also disclosed is a method of determining responsiveness of a heart disease in a subject to treatment with a pharmaceutical agent, comprising a) determining the expression levels of one or more PP2A genes in a first sample from the subject before treatment; b) administering to the subject the pharmaceutical agent; c) determining the expression levels of the corresponding PP2A genes in a second sample from the subject after treatment; and d) comparing the expression levels of the one or more PP2A genes in the second sample from the subject to the expression levels of the corresponding PP2A genes in the first sample from the subject, wherein the normalization of the expression levels of PP2A genes in the second sample from the subject indicates responsiveness of the heart disease to the pharmaceutical agent.

Also disclosed is a method of screening the efficacy of a pharmaceutical agent for treating heart disease in a subject, comprising a) determining the expression levels of one or more PP2A genes in a first sample from the subject before treatment; b) administering to the subject the pharmaceutical agent; c) determining the expression levels of the corresponding PP2A genes in a second sample from the subject after treatment; and d) comparing the expression levels of the one or more PP2A genes in the second sample from the subject to the expression levels of the corresponding PP2A genes in the first sample from the subject, wherein the normalization of the expression levels of PP2A genes in the second sample from the subject indicates efficacy of the pharmaceutical agent.

As described herein, disclosed are methods of detecting heart disease in a sample comprising determining the expression level of one or more genes in a sample and comparing those expression levels to the expression levels of a normal control sample, wherein the expression level of one or more genes or peptides is increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” control sample is indicative of a heart disease. In addition, the expression level of one or more genes or peptides can be increased or decreased by 10%, 15%, 20%, 25%, 30%, 35%, 40%, 45%, 50%, 55%, 60%, 65%, 70%, 75%, 80%, 85%, 90%, 95%, or 100% when compared to the expression level of a “normal” control sample is indicative of heart disease.

Another aspect of the invention relates to a method for diagnosing whether a subject has an increased likelihood of having heart disease, the method comprising determining the level of expression of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 in the subject, in heart tissue of the subject or in a biological sample obtained from the subject, and comparing to the level of expression in an appropriate reference sample or normal control sample, wherein a reduction or increase of the level as specified is indicative of the subject having an increased risk of having heart disease at the time the method is performed.

When the subject is identified to be at risk of developing heart disease using the methods as disclosed herein, the subject may develop the heart disease in the near future or anytime in the future. Accordingly, such subjects can be selected for frequent follow up measurements of the levels of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4, to allow early treatment of the heart disease. Alternatively, the present invention provides methods to diagnose subjects who are at a lesser risk of developing heart disease by analyzing the levels of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 to identify subjects at no or minimal risk of heart disease, who can be selected to undergo less frequent follow up measurements of the level, or other alternative invasive or non-invasive diagnostic methods.

An increase or decrease in the expression level of the genes or peptides disclosed herein is not always required to indicate heart disease. There can be signature patterns of increased or decreased expression levels of one or more of the genes or peptides.

The variance in the obtained profile of expression levels of the said selected genes or gene expression products in said subject's sample can be used to determine the type of treatment, or combination of treatments, that the subject should receive. Examples of treatments typically given to subjects diagnosed with heart disease include, but are not limited to, aspirin, diuretics, calcium channel inhibitors, antiarrhythmics, catecholamines and catecholamine receptor agonists and antagonists (including α- and β-adrenergic receptor agonists and antagonists), vasopressin receptor agonists and antagonists, organic nitrates, activators of soluble and particulate guanylyl cyclases, natriuretic peptides or other components that bind to natriuretic peptide receptors and stimulate or inhibit guanylyl cyclase activity, renin inhibitors, angiotensin-converting enzymes inhibitors, and angiotensin receptor antagonists.

Also disclosed is a method of identifying a subject appropriate for a heart disease treatment clinical trial comprising determining in a sample from the subject the expression levels of one or more PP2A genes, wherein the expression levels of the one or more PP2A genes is indicative of heart disease, indicates whether the subject is appropriate for the treatment clinical trial. The expression levels of one or more PP2A genes can be determined by amplifying a nucleic acid sample obtained from the subject. An increase in the expression levels of PPP2CA or PPP2CB in the subject compared to the expression levels of PPP2CA or PPP2CB in a normal control can indicate the subject is appropriate for the treatment clinical trial.

Also disclosed is a method wherein an increase in the expression levels of PPP2R1A or PPP2R1B in the subject compared to the expression levels of PPP2R1A or PPP2R1B in the normal control, indicates the subject is appropriate for the treatment clinical trial.

An increased expression level of one or more of PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, and PPP2R4 in the subject compared to the expression levels of the corresponding genes in the normal control can indicate the subject is appropriate for the treatment clinical trial. A decreased expression level of one or more of PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, and PPP2R4 in the subject compared to the expression levels of the corresponding genes in the normal control can indicate the subject is appropriate for the treatment clinical trial.

Also disclosed is a method of identifying a subject appropriate for a heart disease treatment clinical trial comprising determining in a sample from a subject an increased ratio of phosphorylated catalytic subunit genes to total catalytic subunit genes compared to a ratio of phosphorylated catalytic subunit genes to total catalytic subunit genes in a normal control sample, wherein the increased ratio of phosphorylated catalytic subunit genes to total catalytic subunit genes in the subject indicates that the subject is appropriate for the treatment clinical trial.

Also disclosed is a method of identifying a subject appropriate for a heart disease treatment clinical trial comprising determining in a sample from a subject a decreased ratio of methylated catalytic subunit genes to total catalytic subunit genes compared to a ratio of methylated catalytic subunit genes to total catalytic subunit genes in a normal control sample, wherein the decreased ratio of methylated catalytic subunit genes to total catalytic subunit genes in the subject indicates that the subject is appropriate for the treatment clinical trial.

In one aspect, the methods can be used to ensure patients are identified to participate in treatment clinical trials based upon the likelihood of heart disease and whether subject can benefit from treatment to prevent the development of heart disease. In one aspect, the methods can comprise measuring an initial value of PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4 in a subject before the subject has received treatment. Repeat measurements can then be made over a period of time. For example, and not to be limiting, that period of time can be about 1 day, 2 days, 5 days, 1 week, 2 weeks, 3 weeks, 1 month, 2 months, 3 months, 4 months, 6 months, 9 months, 1 year, or greater than 1 year. If the initial level is elevated relative to the average level in a control population, a significant reduction in level in subsequent measurements can indicate a positive treatment outcome. Likewise, if the initial level of a measure marker is reduced relative to the average in a control population, a significant increase in measured levels relative to the initial level can signal a positive treatment outcome. Subsequently measured levels are considered to have changed significantly relative to initial levels if a subsequent measured level differs by more than one standard deviation from the mean of repeat measurements of the initial level. If monitoring reveals a positive treatment outcome, that indicates a patient that can be chosen to participate in a treatment clinical trial for that particular therapeutic agent or agents. If monitoring reveals a negative treatment outcome, that indicates a patient that should not be chosen to participate in a treatment clinical trial for that particular therapeutic agent or agents.

Methods of determining or identifying subjects eligible to participate in clinical trial treatments can include the steps of enrolling the subject in the clinical trial and administering the treatment to the subject. For example, after identifying a subject appropriate for a heart disease treatment clinical trial, the subject can be administered a heart disease treatment such as but not limited to aspirin, diuretics, calcium channel inhibitors, antiarrhythmics, catecholamines and catecholamine receptor agonists and antagonists (including α- and β-adrenergic receptor agonists and antagonists), vasopressin receptor agonists and antagonists, organic nitrates, activators of soluble and particulate guanylyl cyclases, natriuretic peptides or other components that bind to natriuretic peptide receptors and stimulate or inhibit guanylyl cyclase activity, renin inhibitors, angiotensin-converting enzymes inhibitors, and angiotensin receptor antagonists.

Also disclosed are methods of treating a subject having heart disease comprising diagnosing or detecting heart disease in a subject using any of the methods disclosed herein, and administering a pharmaceutical agent for treating heart disease to a subject having heart disease or ceasing any treatment for heart disease in a subject not having heart disease. The pharmaceutical agent can be any agent known to treat heart disease or can be a pharmaceutical agent that alters PP2A gene expression. For example, pharmaceutical agents determined with the disclosed screening assays can be used in the methods of treating a subject having heart disease.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Defining the Human Cardiac PP2A Phosphatome

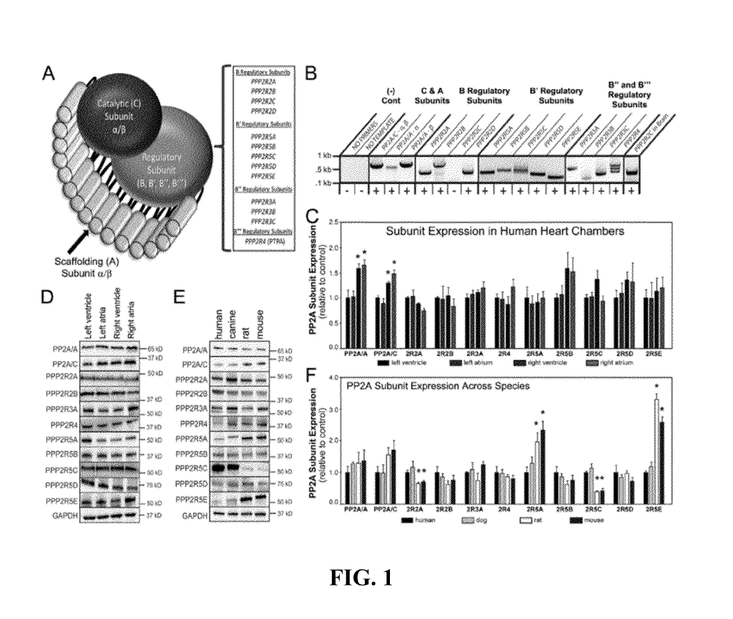

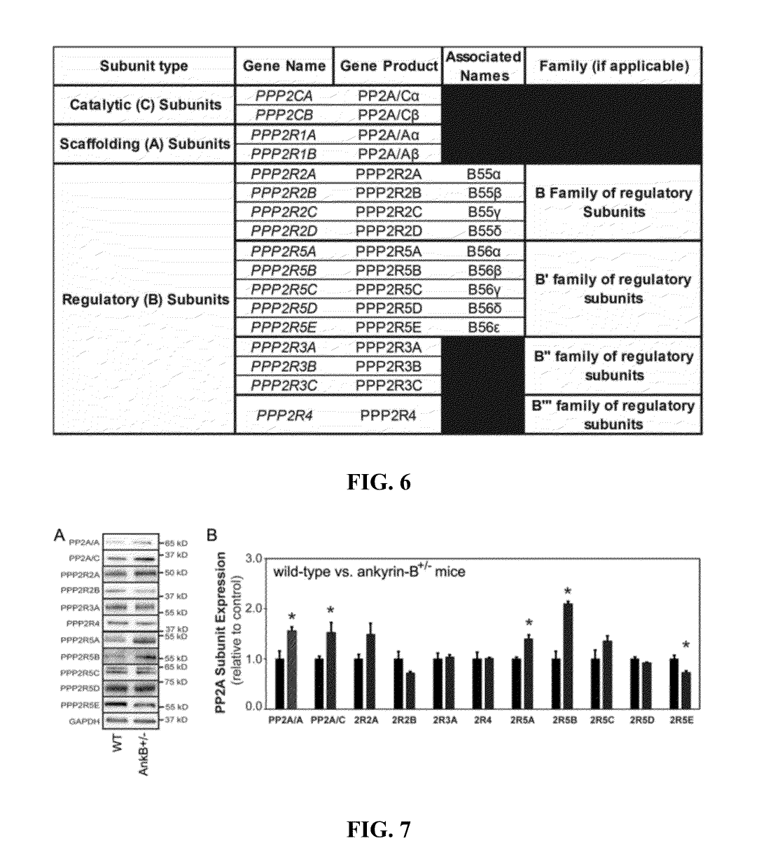

The human genome encodes over seventeen different PP2A subunits (FIG. 1A; 2 A subunits, 13 B subunits, 2 C subunits, FIG. 6 for detailed gene and protein nomenclature of subunit families). However, the prior art does not characterize the expression of the PP2A subunit family in human heart. An analysis of the expression profile of the PP2A subunits in heart was performed to provide important insight into how signaling specificity is achieved for phosphatase activity. PP2A subunits in healthy human heart were first identified using PP2A subunit gene-specific probes. mRNA for both isoforms of the scaffolding subunit (A subunits α and β) in human left ventricle was identified (FIG. 1B). Additionally, message for both the α and β isoforms of the PP2A catalytic subunit was present (FIG. 1B). Notably, while thirteen PP2A regulatory subunit genes are present in the human genome (FIG. 1A), significant mRNA expression of only twelve of thirteen genes in human heart was identified. Specifically, catalytic subunit (PPP2AC), scaffolding subunits (PPP2R1A, PPP2R1B), and regulatory subunits PPP2R2A, PPP2R2B, PPP2R5A, PPP2R5B, PPP2R5C, PPP2R5D, PPP2R5E, PPP2R3A, PPP2R3B, PPP2R3C, and PPP2R4 were detected in human heart cDNA. Message for PPP2R2C, not present in heart, was observed in human brain (FIG. 1B). All appropriate negative controls for PP2A subunit mRNAs were blank.

Human tissue samples. Left ventricular (LV) tissue was obtained from explanted hearts of patients undergoing heart transplantation through The Cooperative Human Tissue Network: Midwestern Division at OSU. Approval for the use of human subjects was obtained from the Institutional Review Board of OSU. LV tissue from healthy donor hearts not suitable for transplantation was obtained through the Iowa Donors Network and the National Disease Research Interchange. The investigation conformed to the principles outlined in the Declaration of Helsinki. Age and sex were the only identifying information acquired from tissue providers.

mRNA analysis. PCR reactions were performed in a volume of 20 μl using Platinum Taq Polymerase High Fidelity. A touchdown PCR protocol was used for each primer set to reduce any non-specific sequence amplification. See Table 1 for primer sequences.

TABLE 1
Primer Sequences

| | | Estimated |
|---|---|---|
| | | PCR product |
| Primer Name | Sequence | size (BP) |
| PP2A/C_5′ | GTT CAG CAA CGA GCT GGA CCA GTG (SEQ ID NO: 1) | 610 |
| PP2A/C_3′ | CCA CCA CGG TCA TCT GGA TCT GAC CA (SEQ ID NO: 2) | |
| PP2A/Aα_5′ | ACC TCT CAG CTG ACT GTC GGG AGA ATG TGA TCA | 505 |
| | TGT CCC (SEQ ID NO: 3) | |
| PP2A/Aα_3′ | CTC CGG ACT GGC CAA GAC CTT GGG GAT GAT TGT | |
| | GGA (SEQ ID NO: 4) | |
| PP2A/Aβ_5′ | CGA TCG CGG TTT TAA TCG ACG AGC TCC GCA ATG | 658 |
| | AAG ACG TGC (SEQ ID NO: 5) | |
| PP2A/Aβ_3′ | ATA CTG ACA CAA GCT TCC ACA GCA AGG AGGG CGC | |
| | ACT GAA TCC (SEQ ID NO: 6) | |
| PPP2R2A_5′ | GCT GGA GGA GGG AAT GAT ATT CAG TGG TGT TTT | 391 |
| | TCT CAG GT (SEQ ID NO: 7) | |
| PPP2R2A_3′ | GTA GGA TCT CTA TAC CTT CCA TCC TCC TCT TTC AAG | |
| | TTA T (SEQ ID NO: 8) | |
| PPP2R2B_5′ | GAT CCT GCC ACC ATC ACA ACC CTG CGG GTG CCT | 747 |
| | (SEQ ID NO: 9) | |
| PPP2R2B_3′ | GTC ACA CAG CCG GAT TGT CCC TTT GCT GCG GC (SEQ | |
| | ID NO: 10) | |
| PPP2R2C_5′ | CDC CDC CCA CTC ACT CCT GTC CAC CAA CGA T | 305 |
| | (SEQ ID NO: 11) | |
| PPP2R2C_3′ | GGA CGG CTT GAT GTC CAC GAT GTT GAA GCT CCT | |
| | GTC GG (SEQ ID NO: 12) | |
| PPP2R2D_5′ | TGG TGC TTC TCG CAG GTC AGG GGG GCC ATC GAC GA | 389 |
| | (SEQ ID NO: 13) | |
| PPP2R2D_3′ | GAC CCG TAG CGC CGT GAT CCT AAA TGG GTC TCG | |
| | AAG TC (SEQ ID NO: 14) | |
| PPP2R5A_5′ | GAG TAT GTT TCA ACT AAT CGT GGT GTA ATT GTT | 303 |
| | GAA TCA GCG (SEQ ID NO: 15) | |
| PPP2R5A_3′ | TCC CAT AAA TTC GGT GCA GAA CAG TCT TCA GG (SEQ | |
| | ID NO: 16) | |
| PPP2R5B_5′ | ATG GAG ACG AAG CTG CCC CCT GCA AGC ACC CCC | 375 |
| | ACT AGC CCC TCC TCC (SEQ ID NO: 17) | |
| PPP2R5B_3′ | GAC GGG CTC GAT GAG GAC ACC CCG GGT GCT CCC | |
| | CAC ACT (SEQ ID NO: 18) | |
| PPP2R5C_5′ | CAG TGA CAA CGC AGC GAA GAT TCT GCC CAT CAT | 347 |
| | (SEQ ID NO: 19) | |
| PPP2R5C_3′ | CTA GCG GCC GTC CTG GGA GGC CAG CTC ATC GGC CC | |
| | (SEQ ID NO: 20) | |
| PPP2R5D_5′ | GGC CCG GCT TAA TCC CCA GTA TCC CAT GTT CCG | 232 |
| | AGC CCC TCC (SEQ ID NO: 21) | |
| PPP2R5D_3′ | TCA GAG AGC CTC CTG GCT GGC AGT TAG GAA CTC | |
| | TTC CGC CCG (SEQ ID NO: 22) | |
| PPP2R5E_5′ | CCG GCT ATT GTG GCG TTG GTG TAC AAT GTG TTG | 203 |
| | AAG GC (SEQ ID NO: 23) | |
| PPP2R5E_3′ | TAA AGT TGG AAT TAT TCC ATC ACG TCT ACG TCT | |
| | AAG ACC TCT CTT TAA (SEQ ID NO: 24) | |
| PPP2R3A_5′ | GGA TGT GGT GGA TAC CCA CCC TGG TCT CAC GTT | 519 |
| | CCT (SEQ ID NO: 25) | |
| PPP2R3A_3′ | CTC ATA CAT GGA GAG TAC ACC GTC TCC ATC CAC | |
| | ATC CAT (SEQ ID NO: 26) | |
| PPP2R3B_5′ | ATG CCG CCC GGC AAA GTG CTG CAG CCG GTC CTG | 280 |
| | (SEQ ID NO: 27) | |
| PPP2R3B_3′ | CGT TCG TGG GGC TGG AGG CGG CGC CAA GGG (SEQ | |
| | ID NO: 28) | |
| PPP2R3C_5′ | TCG TCG GCG CCT AGC GAC GCC CAA CAC CTG (SEQ ID | 320 |
| | NO: 29) | |
| PPP2R3C_3′ | ATC GCT TCC TCT CCA ATC ATA GGT GGT GTC TGG | |
| | TGT TTG TCC AGC (SEQ ID NO: 30) | |
| PPP2R4_5′ | GCT GAG GGC GAG CGG CAG CCG CCG CCA (SEQ ID NO: | 487 |
| | 31) | |
| PPP2R4_3′ | GCC AGA TGG GTA GGG ACC ACT GTG GCC ACC (SEQ ID | |
| | NO: 32) | |

### Example 2

### Expression of PP2A Subunits in Human Heart

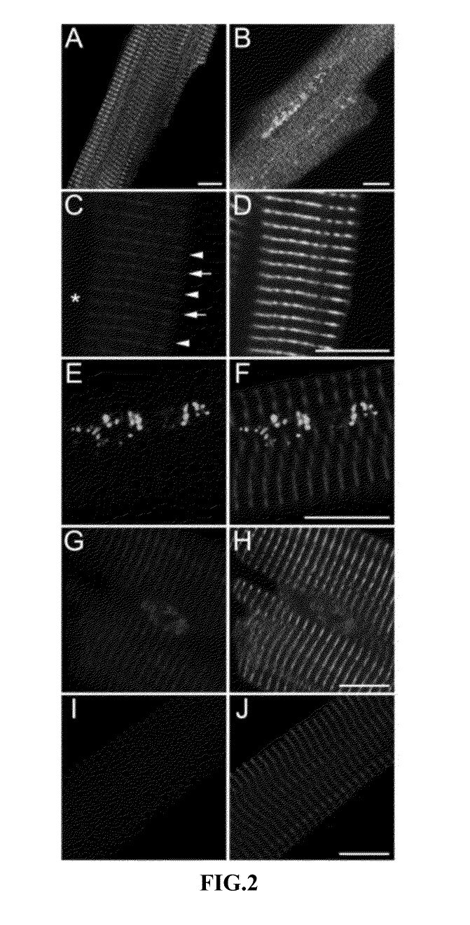

PP2A subunit gene product expression in human heart was also tested. Specifically, the protein expression of catalytic, regulatory, and scaffolding subunits was tested by mRNA (FIG. 1B) in human LV using subtype-specific antibodies. Both PP2A catalytic and scaffolding subunits were observed at the appropriate molecular weights in human LV (FIG. 1C). Nine different PP2A regulatory subunits (PPP2R2A, PPP2R2B, PPP2R3A, PPP2R4, PPP2R5A, PPP2R5B, PPP2R5C, PPP2R5D, and PPP2R5E) were observed at their appropriate molecular weights by immunoblot (FIG. 1C). Other subunits were not detected. Notably, subsequent immunoblot analysis across human heart chambers (LV, RV, LA, RA) revealed differences in PP2A subunit expression. For example, both PP2A catalytic and scaffolding subunit expression were significantly higher in right atria and ventricle when compared with left atria and ventricle (FIG. 2B, C). However, for regulatory subunits, no statistical differences in relative protein expression were observed between the four human heart chambers.

Finally, whether subtype expression was conserved across species was tested. Immunoblots of human, canine, rat, and mouse LV confirmed the presence of both the PP2A catalytic and scaffolding subunits (PP2A-A, PP2A-C) as well as regulatory subunits PPP2R2A, PPP2R2B, PPP2R3A, PPP2R4, PPP2R5A, PPP2R5B, PPP2R5C, PPP2R5D, and PPP2R5E (FIG. 1D). While differences in the expression of the catalytic or scaffolding subunits were not observed, several regulatory subunits were expressed at different levels. PPP2R5A and PPP2R5E expression levels were significantly higher in rat and mouse than in human and dog (FIG. 1D), and PPP2R2A and PPP2R5C were significantly lower in rat and mouse than in human and dog (FIG. 1D). These new findings illustrate the complexity and diversity of PP2A subunit expression across vertebrate heart. Furthermore, they indicate that variable expression of the regulatory subunits confers signaling specificity across regions, species, and disease states.

Immunoblots. Following quantification, tissue lysates were analyzed on Mini-PROTEAN tetra cell (BioRad) on a 4-15% precast TGX gel (BioRad). Gels were transferred to a nitrocellulose membrane using the Mini-PROTEAN tetra cell (BioRad). Membranes were blocked for 1 hour at room temperature using a 3% BSA solution and incubated with primary antibody overnight at 4° C. Antibodies included: anti-PP2A-C subunit (1:500, Millipore 05-421), anti-PPP2R5E (1:1000, Sigma HPA006034), anti-PP2A subunit B isoform PR55α (1:500, Sigma SAB4200241), anti-PP2A subunit B isoform B56-δ (Sigma SAB4200255), anti-PPP2R5A (1:500, Abcam ab72028) or anti-PP2A-B56-α (1:200, Santa Cruz sc-136045), anti-PPP2R5C (1:500 Abcam ab94633), anti-PP2A alpha phospho Y307 (1:500 Abcam, ab32104), anti-PP2A methyl L309 (1:500, Abcam ab66597), anti-PPP2R5B (Abcam ab1366), anti-PPP2R3A (Sigma HPA035829), and anti-PP2A/A (Calbiochem 539509). Secondary antibodies included donkey-anti-mouse-HRP, donkey-anti-rabbit-HRP, and donkey-anti-goat-HRP (Jackson Laboratories). Densitometry was performed using Adobe Photoshop software and all data were normalized to GAPDH levels present in each sample.

Tissue preparation. Cardiac tissue from human, canine, and murine hearts was flash frozen with liquid N₂ and ground into a fine powder using a chilled mortar and pestle. The resulting powder was then resuspended in homogenization buffer (1 mM NaHCO₃, 5 mM EDTA, 1 mM EGTA, 2 mM Na₃VO₄, 1 mMNaF, 1 mM PMSF, and protease inhibitor cocktail [Sigma]) and further homogenized using a chilled Dounce homogenizer. Samples were flash frozen in liquid N₂ and stored at −80° C. for immunoblots.

### Example 3

### Differential Subcellular Expression of PP2A Regulatory Subunits

Whether a secondary level of target specificity arises through the subcellular distribution of specific PP2A holoenzymes was tested by evaluating the subcellular localization of PP2A subunits in primary adult cardiomyocytes. Broad cellular distribution of PP2A scaffolding and catalytic subunits throughout the cytosol and nucleus of ventricular cardiomyocytes was observed. In fact, immunostaining for both the A and C subunits was present across the entire myocyte (FIG. 2A-B). In contrast, specificity in the localization of PP2A regulatory subunits in ventricular myocytes was observed. For example, PPP2R5E was localized specifically to the Z-line/T-tubule region as demonstrated by co-distribution with α-actinin (FIG. 2I-J), while PPP2R3A was localized to both Z- and M-lines of the cardiomyocyte (FIG. 2C-D). PPP2R5C was primarily localized to the myocyte nucleus, with a secondary population overlying the cardiac Z-line (FIG. 2G-H). Finally, PPP2R4 was concentrated in the cardiomyocyte nucleus and localized with nuclear speckles (FIG. 2E-F). Similar results were observed for adult and neonatal primary myocytes. Together, these findings illustrate an additional layer of complexity for regulating PP2A holoenzyme function for specific subcellular targets.

Immunofluorescence. Isolated cardiomyocytes were blocked in PBS, containing 0.075% Triton X-100 and 3% BSA, and were incubated in primary antibody overnight at 4° C. Following washes (PBS plus 0.1% Triton X-100), the slides were incubated in secondary antibody (Alexa 488, or 568) for 4 hours at 4° C. and mounted using Vectashield (Vector) and coverslips. Images were collected on a Zeiss 510 Meta confocal microscope (40 power oil 1.40 NA (Zeiss), pinhole equal to 1.0 Airy Disc) using Carl Zeiss Imaging software. Images were imported into Adobe Photoshop for cropping and linear contrast adjustment.

Cardiomyocyte preparations. Neonatal and adult cardiomyocytes were prepared as previously described [27, 20].

### Example 4

### PP2A Subunit Regulation in Human Heart Failure

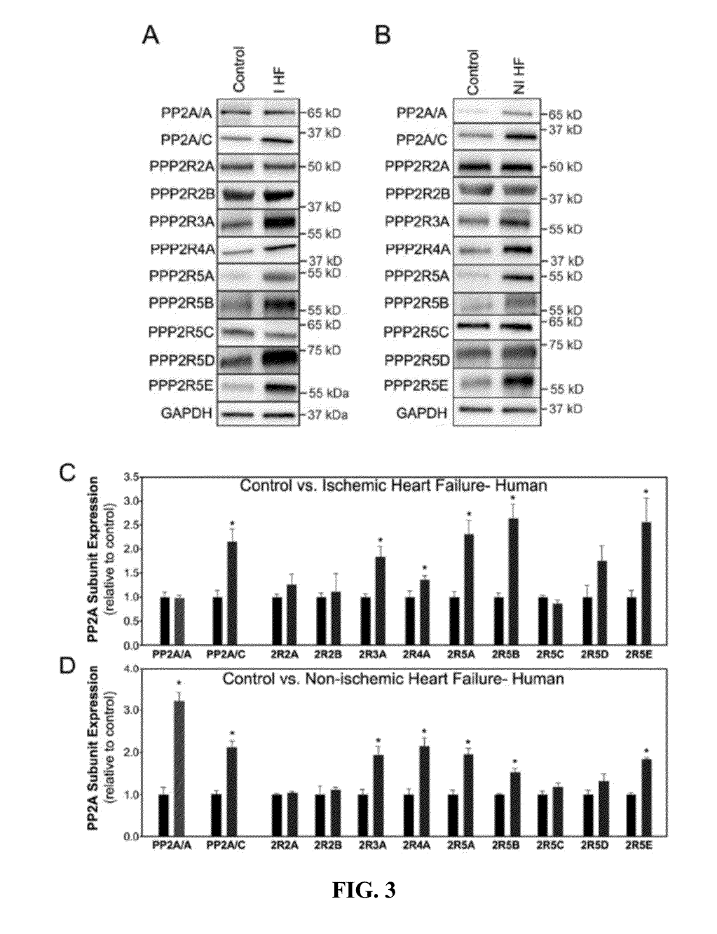

Whether the expression levels of PP2A subunits were altered in multiple human heart failure pathologies was determined. In particular, whether variability in the expression of the regulatory subunits serves as a molecular identifier for disease was determined PP2A subunit expression in human ischemic heart failure samples was first determined. While PP2A scaffolding subunit levels were unaffected in LV tissue, nearly a two fold increase in PP2A catalytic subunit expression, when compared to control hearts, was observed (n=5 control and non-ischemic, p<0.05). Differential regulation of regulatory subunits in ischemic heart failure was also observed, with elevated levels of PPP2R5A, PPP2R5B, PPP2R5E, PPP2R3A, and PPP2R4 (FIG. 3; n=5 control and non-ischemic, p<0.05). Other subunit levels were not statistically different between control and diseased hearts (n=5 control and non-ischemic, N.S.).

PP2A subunit expression was examined in LV samples from both non-ischemic and control hearts. While similar data for PP2A catalytic subunit (increased approximately two fold) were observed, PP2A scaffolding subunit levels were also elevated in non-ischemic LV (n=5 control and non-ischemic, p<0.05 for A, C subunits). Regulatory subunit overexpression was observed for PPP2R5A, PPP2R5B, PPP2R5E, PPP2R3A, and PPP2R4, similar to ischemic heart failure data (FIG. 3B; n=5 control and non-ischemic, p<0.05). Consistent with ischemic heart failure data, no difference in the expression of PPP2R2A, PPP2R2B, PPP2R5C, or PPP2R5D gene products was observed (n=5 control and non-ischemic, N.S.). These data provide the first insight into the complex regulation of PP2A subtype family proteins in human heart disease and clearly demonstrate significant complexity in the regulation of specific PP2A holoenzymes in specific pathologies. These data are also in line with differential target phospho-protein regulation in specific disease pathologies.

### Example 5

### PP2A Subunit Regulation in Canine Cardiovascular Disease Models

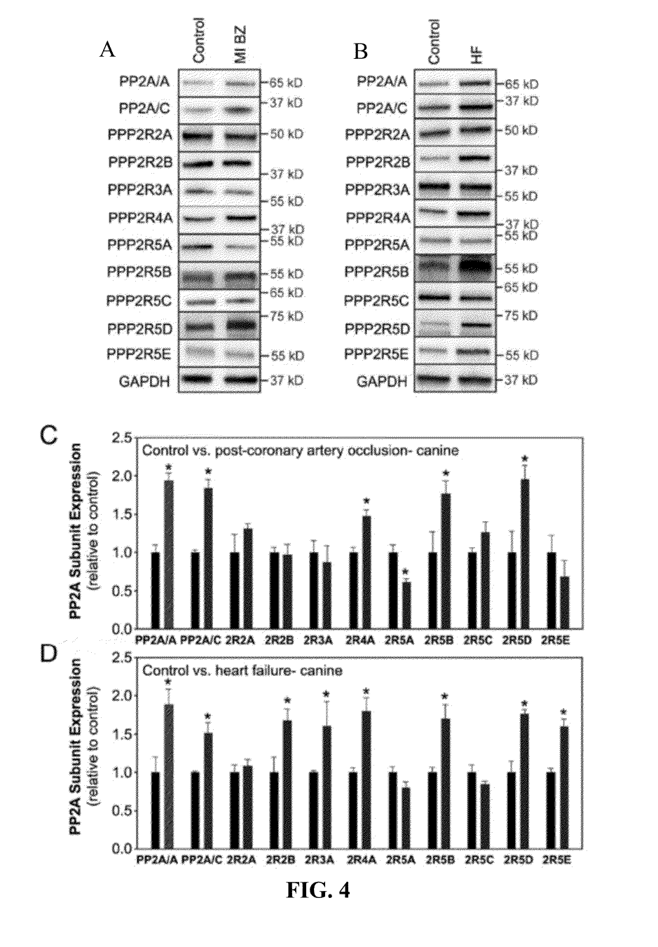

PP2A subunit expression in two different canine models of human heart disease was examined PP2A subunit expression following myocardial infarction (through coronary artery occlusion), a leading cause of death worldwide, was first evaluated [23]. Similar to both ischemic and non-ischemic human heart failure samples, elevated levels of PP2A catalytic subunit were observed in infarct border zone tissue compared to remote tissue of a well validated canine myocardial infarction model [24] (FIG. 4A; n=5 control and post-occlusion, p<0.05). Additionally, PP2A scaffolding subunit levels were also significantly elevated, although only moderately at five days post-occlusion (FIG. 4A; n=5 control and post-occlusion, p<0.05). PP2A regulatory subunits with altered expression included PPP2R5A, PPP2R5D, and PPP2R4 at five days post-injury. At this early time point, no difference in the expression of regulatory subunit products of PPP2R2A, PPP2R2B, PPP2R5C, PPP2R5E, or PPP2R3A was observed (FIG. 4A; n=5 control and post-occlusion, N.S.).

In addition to the canine MI model above, PP2A subunit expression was also evaluated in a well validated long-term tachy-pacing induced non-ischemic model of canine heart failure [22]. At four months of chronic pacing, elevated levels of both the scaffolding and catalytic subunits were observed in experimental samples compared with control tissue (FIG. 4B; n=5 control and HF, p<0.05). PPP2R2B, PPP2R5B, PPP2R5D, PPP2R5E, and PPP2R4 gene products were significantly elevated in failing dog LV compared with control (FIG. 4B; n=5 control and HF, p<0.05). In contrast, levels of PPP2R2A, PPP2R5A, PPP2R5C, and PPP2R3A were unchanged between control and heart failure samples (FIG. 4B; n=5 control and HF, N.S.). Collectively, data in canine ischemic and non-ischemic disease models demonstrate significant regulation of the PP2A enzyme through regulation of PP2A catalytic and scaffolding subunits.

Canine ischemic and non-ischemic heart failure models. Myocardial infarction was produced in healthy mongrel dogs by total coronary artery occlusion, as described previously [20]. A cardiectomy was performed five days after surgery. Thin tissue slices from visible epicardial border zone (BZ) and from a remote area away from the infarct (LVbase) were flash frozen for analysis. HF via chronic tachypacing was induced as described [21, 22]. Cardiac tissues were snap frozen in liquid nitrogen and stored at −80° C. until used. Control dogs were sacrificed in parallel.

### Example 6

### PP2A Subunit Regulation in Mouse Model of Human Catecholaminergic-Induced Arrhythmia

Over the past decade, dysregulation in cardiac sympathetic tone has been linked to both genetic and acquired forms of human ventricular arrhythmia [14]. In fact, mutations in select myocyte pathways that alter the phospho-protein axis have been identified as a primary cause of multiple forms of congenital catecholaminergic polymorphic ventricular tachycardia (CPVT) [25]. One notable pathway altered in human CPVT, sinus node disease, heart rate variability, and atrial fibrillation is the ankyrin-B-pathway that coordinates the subcellular localization of membrane-associated ion channels, transporters, and signaling molecules [26-29]. Whether PP2A subunit levels were altered in an animal model for human ankyrin-B CPVT was examined Consistent with investigated forms of human and canine heart disease (FIG. 2-3), significant increases were observed in both the PP2A catalytic and scaffolding subunits in adult ankyrin-B^{+/-}LV compared with littermates (FIG. 7; n=5 control and ankyrin-B^{+/-}, p<0.05). Additionally, alterations in the expression of PP2A regulatory subunits PPP2R5A, PPP2R5B, and PPP2R5E were observed in ankyrin-B deficient hearts (FIG. 7; n=5 control and ankyrin-B^{+/-}, p<0.05). While there were trends for differences in other PP2A regulatory subunits, no significant differences in the expression of PPP2R2A, PPP2R2B, PPP2R3A, PPP2R4, PPP2R5C, or PPP2R5D were observed between WT and ankyrin-B^{+/-} mice (FIG. 7; n=5 control and ankyrin-B^{+/-}, p<0.05).

Mouse heart failure model with proximal aortic banding. Adult male B6 mice were subjected to pressure overload by TAC surgery as previously described [21].

### Example 7

### Post-Translational Regulation of Cardiac PP2A Function in Human Cardiovascular Disease

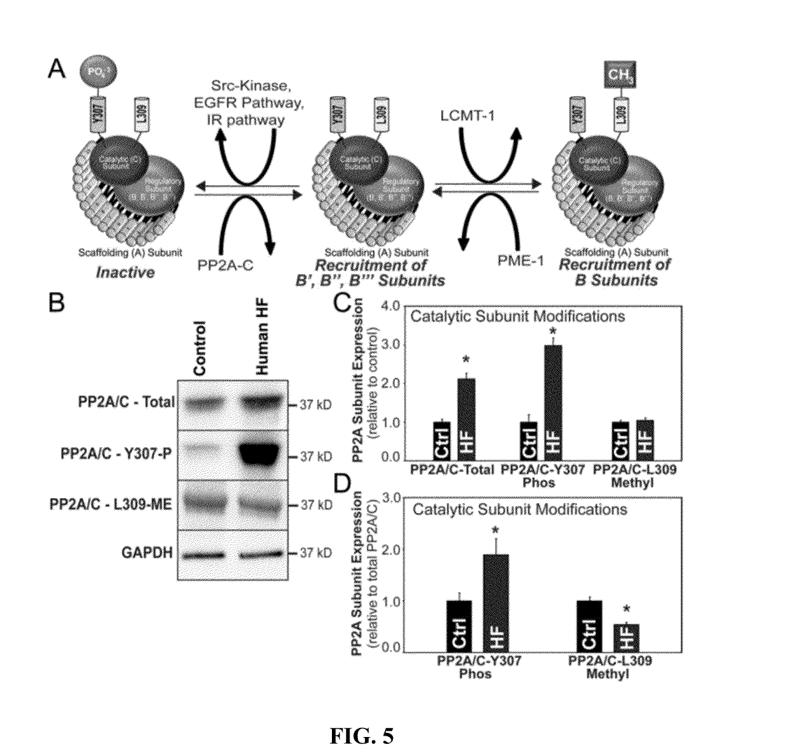

While tissue, cell, and subcellular subunit expression and localization play key roles in determining local holoenzyme function, post-translational regulation of PP2A activity has been implicated in holoenzyme regulation in other organ systems and non-cardiac disease states [9, 30]. Notably, the C-terminus of the PP2A catalytic subunit interacts with an interface of the A and B subunits. This C-terminal region is altered by both phosphorylation and methylation that alters recruitment and docking of the regulatory subunit with the A and C subunits to form the active holoenzyme (FIG. 5A) [30]. Whether these modifications were present in human heart and modified in human heart disease was examined.

The phosphorylation status of the catalytic subunit of PP2A at residue Y307, a site liked with inactivation of the phosphatase by pp60^{vSrc}, pp56^{1ck}, epidermal growth factor, and insulin receptor signaling at baseline in heart, and in heart disease was first examined [31]. Notably, phosphorylated PP2A/C Y307 was observed at baseline in human heart (FIG. 5). Moreover, a significant increase was observed in the expression level of the phosphorylated (inactivated) catalytic subunit in heart failure samples compared to controls (FIG. 5; p<0.05, n=4). In contrast, no difference was observed in the methylation of PP2A catalytic subunit at residue L309, regulated by the opposing activities of leucinecarboxymethyl-transferase 1 (LCMT-1) and phosphatase methyl-transferase 1 (PME1) to control recruitment of the PPP2R2A and PPP2R2B regulatory subunits [32]. Total PP2A catalytic subunit expression was increased in non-ischemic heart failure (FIG. 5). However, in parallel, levels of PP2A-phospho-Y307 (inactivated) were increased. Therefore, expression of phosphorylated and methylated forms of the PP2A catalytic subunit was normalized to the expression level of the total amount of the PP2A/C subunit expressed in each sample. Importantly, these results demonstrated that the ratio of phosphorylated catalytic subunit to total catalytic subunit was increased in human heart failure samples compared to control (FIG. 5; p<0.05; n=4), whereas the ratio of methylated catalytic subunit to total catalytic subunit was significantly decreased in human heart failure compared to control tissue (FIG. 5; p<0.05). In summary, these findings provide the first data on PP2A subunit post-translational regulation in heart, and provide data on the regulation of these critical subunits in human heart failure. Both translational pathways identified in heart failure inactivate the holoenzyme.

### REFERENCES

1. He B J, et al. (2011) Oxidation of CaMKII determines the cardiotoxic effects of aldosterone. Nature Medicine 17(12): 1610-1618.
2. Chelu M G, et al. (2009) Calmodulin kinase II-mediated sarcoplasmic reticulum Ca2+ leak promotes atrial fibrillation in mice. J Clin Invest 119(7): 1940-1951.
3. Swaminathan P D, et al. (2011) Oxidized CaMKII causes cardiac sinus node dysfunction in mice. J Clin Invest 121(8): 3277-3288.
4. Reiken S, et al. (2001) Beta-adrenergic receptor blockers restore cardiac calcium release channel (ryanodine receptor) structure and function in heart failure. Circulation 104(23): 2843-2848.
5. Shan J, et al. (2010) Phosphorylation of the ryanodine receptor mediates the cardiac fight or flight response in mice. J Clin Invest 120(12): 4388-4398.
6. Packer M. (1985) Sudden unexpected death in patients with congestive heart failure: a second frontier. Circulation 72(4): 681-685.
7. Reiken S, et al. (2003) Beta-blockers restore calcium release channel function and improve cardiac muscle performance in human heart failure. Circulation 107(19): 2459-2466.
8. Shelton R J, et al. (2009) A randomised, controlled study of rate versus rhythm control in patients with chronic atrial fibrillation and heart failure: (CAFE-II Study). Heart (British Cardiac Society) 95(11): 924-930.
9. Janssens V & Goris J. (2001) Protein phosphatase 2A: a highly regulated family of serine/threonine phosphatases implicated in cell growth and signalling. Biochemical J 353(Pt 3): 417-439.
10. Eichhorn P J, Creyghton M P, & Bernards R. (2009) Protein phosphatase 2A regulatory subunits and cancer. Biochimica et Biophysica Acta 1795(1): 1-15.
11. Rudrabhatla P & Pant H C. (2011) Role of protein phosphatase 2A in Alzheimer's disease. Curr Alzheimer Res 8(6): 623-632.
12. Ai X & Pogwizd S M. (2005) Connexin 43 downregulation and dephosphorylation in nonischemic heart failure is associated with enhanced colocalized protein phosphatase type 2A. Circ Res 96(1): 54-63.
13. Xu H, et al. (2010) Targeting of protein phosphatases PP2A and PP2B to the C-terminus of the L-type calcium channel Ca v1.2. Biochemistry 49(48):10298-10307.
14. Marx S O, et al. (2000) PKA phosphorylation dissociates FKBP12.6 from the calcium release channel (ryanodine receptor): defective regulation in failing hearts. Cell 101(4): 365-376.
15. Deshmukh P A, Blunt B C, & Hofmann P A. (2007) Acute modulation of PP2A and troponin I phosphorylation in ventricular myocytes: studies with a novel PP2A peptide inhibitor. Am J Physiol Heart Circ Physiol 292(2): H792-799.
16. Wijnker P J, et al. (2011) Protein phosphatase 2A affects myofilament contractility in non-failing but not in failing human myocardium. J Muscle Res Cell Motility 32(3): 221-233.
17. Schulze D H, Mughal M, Lederer W J, & Ruknudin A M. (2003) Sodium/calcium exchanger (NCX1) macromolecular complex. J Biol Chem 278(31): 28849-28855.
18. Kohr M J, Davis J P, & Ziolo M T. (2009) Peroxynitrite increases protein phosphatase activity and promotes the interaction of phospholamban with protein phosphatase 2A in the myocardium. Nitric Oxide 20(3): 217-221.
19. Gergs U, et al. (2004) Overexpression of the catalytic subunit of protein phosphatase 2A impairs cardiac function. J Biol Chem 279(39): 40827-40834.
20. Hund T J, et al. (2009) Regulation of the ankyrin-B-based targeting pathway following myocardial infarction. Cardiovasc Res 81(4): 742-749.
21. Gudmundsson H, et al. (2012) Differential regulation of EHD3 in human and mammalian heart failure. J Mol Cell Cardiol 52(5): 1183-1190.
22. Nishijima Y, et al. (2005) Canine nonischemic left ventricular dysfunction: a model of chronic human cardiomyopathy. J Cardiac Failure 11(8): 638-644.
23. Roger V L, et al. (2012) Executive summary: heart disease and stroke statistics—2012 update: a report from the American Heart Association. Circulation 125(1): 188-197.
24. Pu J & Boyden P A. (1997) Alterations of Na+ currents in myocytes from epicardial border zone of the infarcted heart. A possible ionic mechanism for reduced excitability and postrepolarization refractoriness. Circ Res 81(1): 110-119.
25. Lehnart S E, et al. (2007) Inherited arrhythmias: a National Heart, Lung, and Blood Institute and Office of Rare Diseases workshop consensus report about the diagnosis, phenotyping, molecular mechanisms, and therapeutic approaches for primary cardiomyopathies of gene mutations affecting ion channel function. Circulation 116(20): 2325-2345.
26. Cunha S R, et al. (2011) Defects in ankyrin-based membrane protein targeting pathways underlie atrial fibrillation. Circulation 124(11): 1212-1222.
27. Mohler P J, et al. (2003) Ankyrin-B mutation causes type 4 long-QT cardiac arrhythmia and sudden cardiac death. Nature 421(6923): 634-639.
28. Le Scouarnec S, et al. (2008) Dysfunction in ankyrin-B-dependent ion channel and transporter targeting causes human sinus node disease. P Natl Acad Sci USA 105(40): 15617-15622.
29. Cunha S R, Bhasin N, & Mohler P J. (2007) Targeting and stability of Na/Ca exchanger 1 in cardiomyocytes requires direct interaction with the membrane adaptor ankyrin-B. J Biol Chem 282(7): 4875-4883.
30. Longin S, et al. (2007) Selection of protein phosphatase 2A regulatory subunits is mediated by the C terminus of the catalytic Subunit. J Biol Chem 282(37): 26971-26980.
31. Chen J, Martin B L, & Brautigan D L. (1992) Regulation of protein serine-threonine phosphatase type-2A by tyrosine phosphorylation. Science 257(5074): 1261-1264.
32. Janssens V, Longin S, & Goris J. (2008) PP2A holoenzyme assembly: in cauda venenum (the sting is in the tail). Trends in Biochemical Sciences 33(3): 113-121.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

It will be apparent to those skilled in the art that various modifications and variations can be made in the present invention without departing from the scope or spirit of the invention. Further, those skilled in the art will recognize or be able to ascertain, using no more than routine experimentation, numerous equivalents to the specific embodiments described specifically herein. Such equivalents are intended to be encompassed in the scope of the appended claims Other aspects of the invention will be apparent to those skilled in the art from consideration of the specification and practice of the invention described herein. It is intended that the specification and examples be considered as exemplary only, with a true scope and spirit of the invention being indicated by the following claims.

1. A method of detecting heart disease in a subject, comprising determining in a sample from a subject expression levels of one or more Protein Phosphatase 2A (PP2A) genes and comparing the expression level of the one or more PP2A genes to expression levels of corresponding PP2A genes in a sample from a normal control, wherein an increase or decrease in the expression levels of one or more PP2A genes in the sample from the subject compared to the expression levels of corresponding PP2A genes in the sample from the normal control detects heart disease in the subject.

2. The method of claim 1, wherein the expression level of the one or more Protein Phosphatase 2A (PP2A) genes is determined by amplifying a nucleic acid sample obtained from the subject.

3. The method of claim 1, further comprising administering a therapeutic composition to the subject.

4. The method of claim 1, wherein the PP2A genes are PPP2CA, PPP2CB, PPP2R1A, PPP2R1B, PPP2R2A, PPP2R2B, PPP2R2C, PPP2R2D, PPP2R5A, PPP2R5B, PPP2R5C, PPP2R5D, PPP2R5E, PPP2R3A, PPP2R3B, PPP2R3C, or PPP2R4.

5. (canceled)

6. (canceled)

7. The method of claim 4, wherein an increase in the expression level of PPP2CA and PPP2CB in the subject compared to the expression levels of PPP2CA and PPP2CB in the normal control detects heart disease in the subject.

8-17. (canceled)

18. The method of any of claim 1, wherein the heart disease is ischemic heart disease, non-ischemic heart disease, myocardial infarction, tachy-pacing induced non-ischemic heart disease, or catecholaminergic induced arrhythmia.

19. The method of any of claim 1, wherein the subject is a mammal.

20. The method of claim 19, wherein the mammal is human.

21. The method of any of claim 1, wherein the sample is a body fluid.

22. The method of claim 21, wherein the body fluid is blood, plasma, serum, urine, or saliva.

23. The method of claim 18, wherein the heart disease is ischemic heart disease.

24. The method of claim 23, wherein increased expression levels of PPP2CA, PPP2CB, PPP2R5A, PPP2R5B, PPP2R5E, PPP2R3A, and PPP2R4 compared to the expression levels of the corresponding genes in the normal control detects ischemic heart disease in the subject.

25. (canceled)

26. The method of claim 18, wherein the heart disease is non-ischemic heart disease.

27. The method of claim 26, wherein increased levels of expression of PPP2CA, PPP2CB, PPP2R1A, PPP2R1B, PPP2R5A, PPP2R5B, PPP2R5E, PPP2R3A, and PPP2R4 compared to the expression levels of the corresponding genes in the normal control detect non-ischemic heart disease in the subject.

28. (canceled)

29. The method of claim 18, wherein the heart disease is myocardial infarction.

30. The method of claim 29, wherein increased levels of expression of PPP2CA, PPP2CB, PPP2R1A, PPP2R1B, PPP2R5D, and PPP2R4, and a decreased level of expression of PPP2R5A compared to the expression levels of the corresponding genes in the normal control detect myocardial infarction in the subject.

31. (canceled)

32. The method of claim 18, wherein the heart disease is tachy-pacing induced non-ischemic heart disease.

33. The method of claim 32, wherein increased levels of expression of PPP2CA, PPP2CB, PPP2R1A, PPP2R1B, PPP2R2B, PPP2R5B, PPP2R5D, PPP2R5E, and PPP2R4 compared to the expression levels of the corresponding genes in the normal control detect tachy-pacing induced non-ischemic heart disease in the subject.

34. (canceled)

35. The method of claim 18, wherein the heart disease is catecholaminergic induced arrhythmia.

36. The method of claim 35, wherein increased levels of expression of PPP2CA, PPP2CB, PPP2R1A, PPP2R1B, PPP2R5A, and PPP2R5B, and a decreased level of expression of PPP2R5E compared to the expression levels of the corresponding genes in the normal control detect catecholaminergic induced arrhythmia in the subject.

37-50. (canceled)
